(19) United States
(12) Patent Application Publication
(10) Pub. No.: US 2006/0180157 A1
(43) Pub. Date: Aug. 17, 2006

(54) Foreskin prosthesis

(76) Inventor: James H. Williams, Salisbury (GB)

Correspondence Address: LITMAN LAW OFFICES, LTD, PO BOX 15035, CRYSTAL CITY STATION, ARLINGTON, VA, 22215, US

(21) Appl. No.: 11/402,854

(22) Filed: Apr. 13, 2006

Related U.S. Application Data
(63) Continuation-in-part of application No. 11/111,776, filed on Apr. 22, 2005.

(30) Foreign Application Priority Data
Aug. 18, 2004 (GB) GB0418399.2

(51) Int. Cl. A61F 6/04

(52) U.S. Cl. 128/844

(57) ABSTRACT

The foreskin prosthesis is installed over the penile glans and shaft of a circumcised male and functions similarly to a natural foreskin, protecting the glans from abrasion and desensitization due to contact with clothing. This allows the glans to regain sensitivity previously lost due to constant exposure to abrasion and contact with clothing, thereby improving sexual stimulation and performance. The prosthesis may be retracted to expose the glans, just as a natural foreskin. A series of axial folds may be provided in the glans coverage area, allowing this area to distend circumferentially about the glans to avoid undue restriction during prosthesis retraction. In each embodiment, a single tube is everted back over itself to form two protective layers. In some embodiments, the two layers are sealed to one another at both ends, forming a volume therebetween containing a liquid or gel for further tactile isolation and protection of the glans.

### CROSS-REFERENCE TO RELATED PATENT APPLICATION

This application is a continuation in part of U.S. Utility patent application Ser. No. 11/111,776 filed on Apr. 22, 2005, which application claims the benefit of British Patent Application No. GB0418399.2, filed on Aug. 18, 2004.

### BACKGROUND OF THE INVENTION

1. Field of the Invention

The present invention relates generally to prostheses and artificial anatomical devices. More particularly, the present invention comprises various embodiments of an artificial foreskin for the penis, providing many or most of the benefits of the natural foreskin in the protection of the glans and other functions.

2. Description of the Related Art

Male circumcision is a relatively widely practiced procedure in many societies and cultures. While widely practiced as a religious rite, it has often been carried out for hygienic purposes as well. The procedure is generally performed on infants or very young children, who have no say in the procedure. Nevertheless, the results of the procedure remain with them for the remainder of their lives, and may have some physical and/or psychological impact upon the person. While present medical practice is finding that circumcision is not necessarily a desirable or necessary operation, the fact remains that many men have been circumcised as infants or children, and suffer the effects of the operation to this day.

While a properly performed circumcision does not physically endanger a normal male, the vast number of circumcisions performed has resulted in a statistically significant percentage with occasional severe damage, ranging from amputation of at least some portion of the genitalia to severe infection, and even to death in some cases due to severe infection or hemophilia. While some justification may be made on the basis of hygiene and/or religious grounds, a strong argument may be made that the vast majority of circumcisions performed are unnecessary.

Circumcision can result in a wide array of negative repercussions, as noted further above. One of those repercussions is the permanent exposure of the glans of the penis to abrasion and irritation, whereas in its natural state the glans is covered and protected by the foreskin. In many cases, the glans becomes keratinized and loses sensitivity, which can negatively affect sexual desire and performance. In other cases, the glans remains relatively sensitive to contact (its natural state), and is unduly irritated by contact with clothing and nightwear virtually 24 hours a day.

As a result, a number of artificial or synthetic devices have been developed in the past for the protection of the glans of the penis in circumcised men. Most such devices are formed of relatively coarse or thick materials, which sometimes irritate the glans to about the same extent as underclothing or nightwear. Some of these devices are formed of fabric and include a lateral seam, which, of course, results in an unnatural feel and irritation of the sensitive glans and other surface tissues of the penis. Still other devices have been developed for this purpose, but fail to provide a realistic and natural prosthetic foreskin effect.

The present inventor is aware of certain related art, which discloses devices having loosely related structure and/or function. An example of such is found in Japanese Patent Publication No. 6-165,794 published on Jun. 14, 1994. According to the drawings and English abstract, this device comprises a male catheter, which secures adhesively about the penile shaft behind the glans. The outlet is a very narrow tube adapted to connect to a catheter tube or line. The device is not contoured to fit closely about the coronal sulcus behind the glans, and thus cannot truly emulate a foreskin attachment at that point. Moreover, the configuration of the device of the Japanese Patent Publication as a catheter attachment precludes the retraction of the device to uncover the glans.

None of the inventions and patents of which the present inventor is aware, taken either singly or in combination, is seen to describe the instant invention as claimed. Thus, a foreskin prosthesis solving the aforementioned problems is desired.

### SUMMARY OF THE INVENTION

The foreskin prosthesis provides a thin, soft, pliable covering or sheath for covering the otherwise exposed glans of the penis of a circumcised male. Circumcision often results in irritation and keratinizing of the glans, thereby reducing its sensitivity and ability to be stimulated. The present prosthesis is worn over the penis, and extends from the penile shaft forwardly over the glans to protect the glans and provide a realistic simulation of the natural foreskin.

The prosthesis is open at its distal end, thereby permitting natural urination without interference. The device may be worn continually for a reasonably prolonged period, e.g., for the duration of time between bathing, to provide maximal protection and allow the glans to revert to its natural soft and sensitive state over a period of time. The distal end of the device, i.e., the portion extending over the glans, may be retracted in the same manner as a natural foreskin in an uncircumcised male. In at least some embodiments of the device, it may be provided with a series of thin, pliable axial corrugations or flutes, which distend and stretch circumferentially to facilitate retraction of the device over the penile shaft.

The foreskin prosthesis includes various embodiments comprising different materials and configurations. The device may be formed of a thin, flexible, at least somewhat elastic material, e.g., latex or a synthetic plastic material. Alternatively, the device may be formed as a seamless knitted or braided fabric or textile tube, using a fabric or yarn of sufficiently fine gauge and suitable manufacturing machinery. The device comprises two layers of material, with the inner layer being applied directly over the exposed glans and the outer layer being everted back over the inner layer. The two layers or plies of material greatly reduce the transfer of any sensation or friction from clothing, etc., to the glans. When liquid impervious materials are used, e.g., latex, the device may be formed as two concentric tubes with a liquid or gel contained within the closed concentric walls, thereby further lessening the transfer of tactile sensation to the penile glans through the prosthesis.

Preferably, the diameter of at least the inner sleeve or portion of the device is configured to fit closely about the different diameters of the glans up to the corona of the glans, the sulcus behind the glans, and the penile shaft. This configuration provides a closer simulation of the fit of the natural foreskin. Security for the prosthesis is provided by one or more circumferential bands of a relatively high friction coefficient material, e.g., a soft silicon or other suitable plastic, applied to the base or proximal end of the device. Alternatively, one or more elastomer bands may be applied to the same area, upon the outer and/or inner surface, to provide the desired adhesion and security.

These and other features of the present invention will become readily apparent upon further review of the following specification and drawings.

### BRIEF DESCRIPTION OF THE DRAWINGS

FIG. 1 is a perspective view of a first embodiment of a foreskin prosthesis according to the present invention, showing its various features.

FIG. 2 is a perspective view in section of the extended prosthesis of FIG. 1 before eversion of the outer sleeve, showing details of its construction and configuration.

FIG. 3 is a perspective view in section of the prosthesis of FIGS. 1 and 2, with the outer portion everted over the inner portion in an installed configuration.

FIG. 4 is a perspective view in section of an alternative embodiment of a foreskin prosthesis according to the present invention wherein the two layers define a permanently sealed volume therebetween.

FIG. 5 is a perspective view of the foreskin prosthesis of the present invention, showing the retraction of the forward portion thereof to expose the penile glans.

FIG. 6 is a side view in section of another alternative embodiment of a foreskin prosthesis according to the present invention, wherein only the inner sleeve portion is configured to fit closely about the circumcised penis.

FIG. 7 is a side view in section of the embodiment of FIG. 6, with the outer sleeve everted over the inner sleeve.

FIG. 8 is a perspective view in section showing the two sleeves of the foreskin prosthesis of the present invention rolled in opposite directions for eversion of the outer sleeve over the inner sleeve upon installation.

FIG. 9 is a perspective view similar to that of FIG. 1, in which an alternative embodiment includes a series of axial folds or flutes therein to facilitate the retraction of the device over the glans.

FIG. 10 is a side view in section of the embodiment of FIG. 9, showing the generally medially disposed axial folds or flutes in the device before eversion of the outer sleeve over the inner sleeve.

FIG. 11 is a side view in section of another alternative embodiment based upon the embodiment of FIG. 9, wherein the axial folds are disposed upon the inner sleeve at its juncture with the outer sleeve portion.

FIG. 12 is a side view in section of another alternative embodiment based upon the embodiment of FIG. 9, wherein the axial folds are disposed upon the outer sleeve at its juncture with the inner sleeve portion.

Similar reference characters denote corresponding features consistently throughout the attached drawings.

### DETAILED DESCRIPTION OF THE PREFERRED EMBODIMENTS

The present invention comprises various embodiments of a prosthetic foreskin for use by circumcised males. The naturally occurring foreskin serves as a protective sheath for the underlying glans of the penis, protecting the glans from abrasion and maintaining the tactile sensitivity of the glans organ. Circumcision results in the exposure of the glans to abrasion from clothing, nightwear, etc., and generally reduces its tactile sensitivity. This often results in some loss of sexual desire and capability in many circumcised men. The present foreskin prosthesis serves as a protective cover or sheath for the glans, enabling it to regain much of its previously lost tactile sensitivity and reducing the keratinization that have occurred to the glans surface.

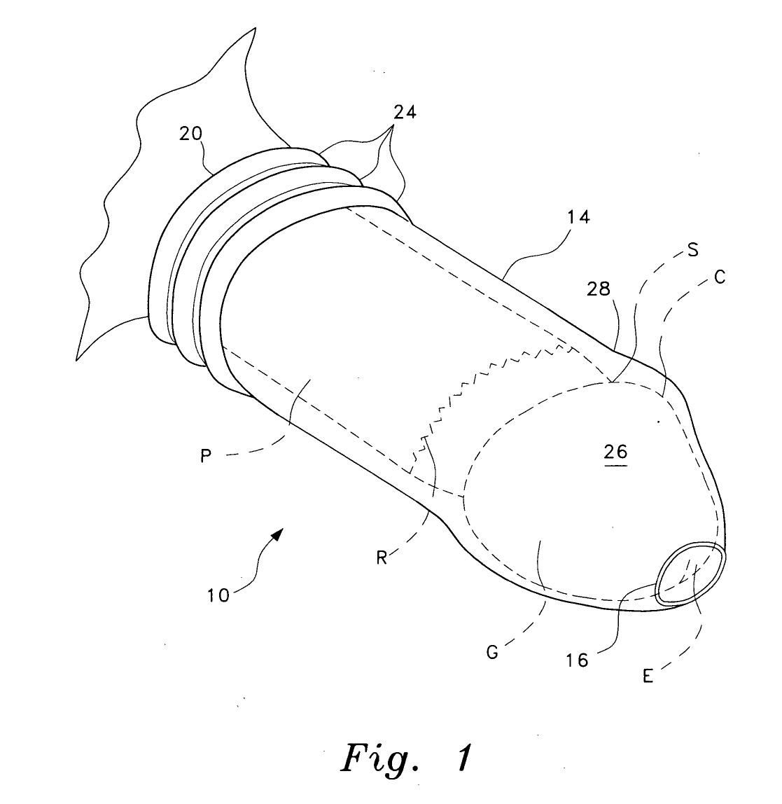

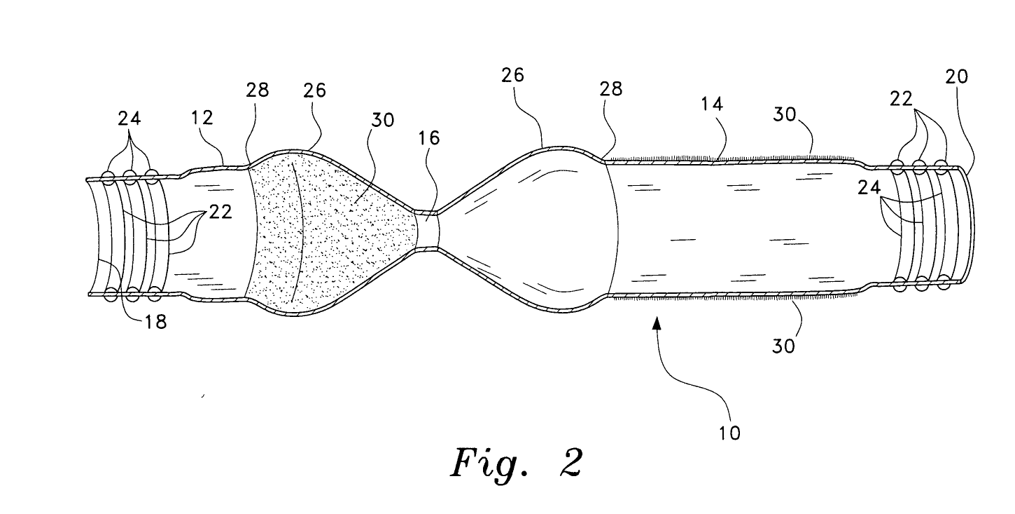

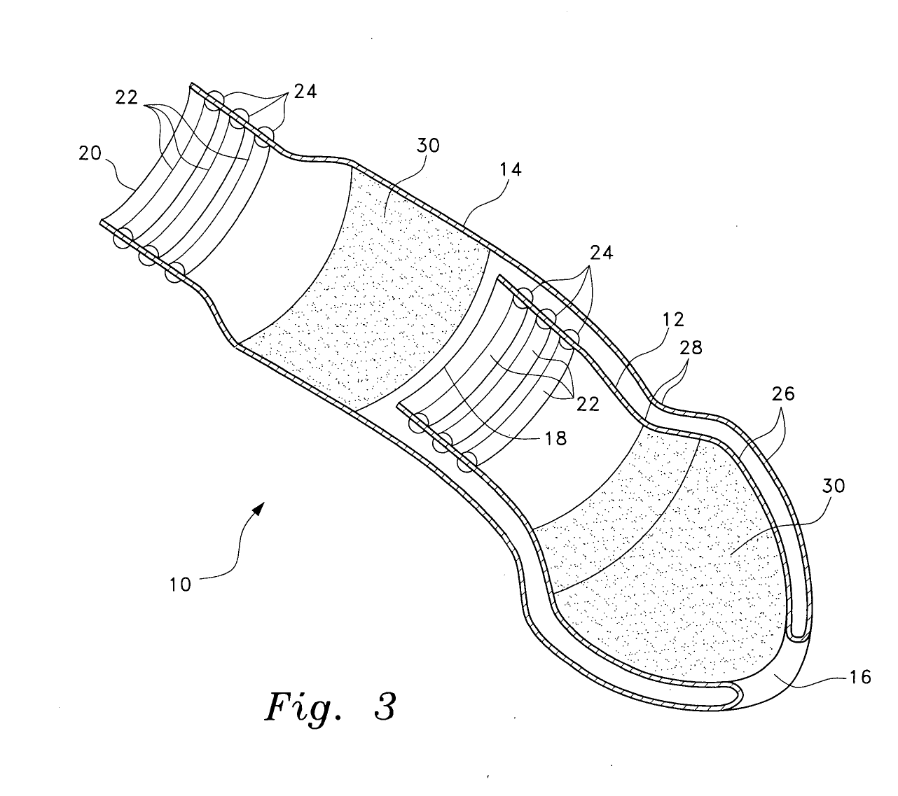

FIG. 1 of the drawings provides a perspective view of a first embodiment of the present foreskin prosthesis 10, with FIGS. 2 and 3 showing the device in section in its extended and installed configurations, respectively. The prosthesis 10 of FIGS. 1 through 3 includes a relatively short inner sleeve 12, shown in FIGS. 2 and 3, configured to extend from the distal end E of the penis P to a point or location immediately behind and adjacent to the coronal sulcus S, i.e., the relatively smaller diameter groove behind the corona C of the glans G, thereby covering and protecting the otherwise exposed glans and penis to a point behind the circumcision scar R. A relatively longer outer sleeve 1 4 extends from the distal end of the penis P substantially to the proximal end or base of the penis P when the device is in its installed configuration with the outer sleeve 14 everted over the inner sleeve 12, as shown in FIGS. 1 and 3. The difference in the lengths of the two sleeves 12 and 14 in the installed configuration of the prosthesis 10 is shown clearly in FIG. 3 of the drawings.

The two sleeves 12 and 14 actually form a single, continuous, seamless tube, as shown in section in FIG. 2 of the drawings. The two sleeves 12 and 14 are joined at their mutual distal end opening 16, which allows urination without need to remove the prosthesis 10 when it is being worn. While the end opening 16 is shown at a generally medial point along the continuous tube in FIG. 2 (depending upon the relative lengths of the two sleeves 12 and 14), the eversion of the outer sleeve 14 over the inner sleeve 12 results in the end opening 16 being positioned at the distal end E of the penis P, when the prosthesis is being worn.

Each sleeve 12 and 14 has an open proximal end 18 and 20, respectively, for the inner and outer sleeves 12 and 14, opposite the mutual distal end opening 16. Some means of securing each of the sleeves 12 and 14 in place about the penile shaft may be provided, e.g., one or more internal or external resilient circumferential securing bands, respectively 22 and 24. (It will be noted that the terms “internal” and “external,” as applied to the two types of bands 22 and 24, refer to their locations on or in the sleeves 12 and 14 when the two sleeves are installed upon the penis in the configuration shown in FIGS. 1 and 3. The non-everted outer sleeve 14 in FIG. 2 shows the bands 22 and 24 reversed relative to their installed locations.)

The internal band(s) 22 may be formed of a very soft and resilient silicone material having a relatively high coefficient of friction in order to provide a secure grip about the penile shaft without undue pressure. The external band(s) 24 may be formed of an elastomer to draw the proximal openings 18 and 20 about the penile shaft. While both types of securing bands 22 and 24 are illustrated in FIGS. 2 and 3, it will be understood that they may be provided exclusively of one another, if so desired. Alternative securing means in the form of a scrotal strap (not shown) may be provided in addition to, or in lieu of, the circumferential bands, if so desired.

Each sleeve 12 and 14 expands in diameter from the end opening 16 to a relatively larger diameter glans coverage area 26, adapted to fit comfortably about the generally larger diameter of the glans G, or at least its larger diameter corona C. The two sleeves 12 and 14 then taper to a smaller diameter sulcus coverage area 28 and may widen slightly in the area therebehind, i.e., the penile shaft.

While a good fit is desired, it is not desired that the present foreskin prosthesis 10 (or other embodiments) fit particularly tightly when being worn. However, excessive looseness is not desired either, as this would allow the device to move relative to the surface of the penis, which would obviate much of the protective benefits of the device. The present foreskin prosthesis in its various embodiments may be formed of various materials, e.g., latex, synthetic plastics, etc., or alternatively of a seamless, finely woven or finely braided fabric tube, as desired. Material that is otherwise impervious may be provided with myriad tiny porosities to enable the underlying skin to “breathe,” if so desired, at least in the embodiment of FIGS. 1 through 3.

While the material used is quite thin, flexible, and resilient, it is not intended to transmit tactile sensation therethrough, as would a condom, but rather teaches away from this property by insulating the penis, and particularly the glans, from pressure, abrasion, and other tactile sensation. The object of the device is to serve in the manner of the natural foreskin, and protect the otherwise exposed glans from clothing and/or other physical contact which lessens its tactile sensitivity and which may result in keratinization of the glans surface. This feature may be further enhanced by the provision of very fine, soft flocking 30 disposed upon the inner contact surface of the glans coverage area of the inner sleeve 12 and perhaps on the inner contact surface of the penile shaft contact area of the outer sleeve 14, as shown in FIGS. 2 and 3.

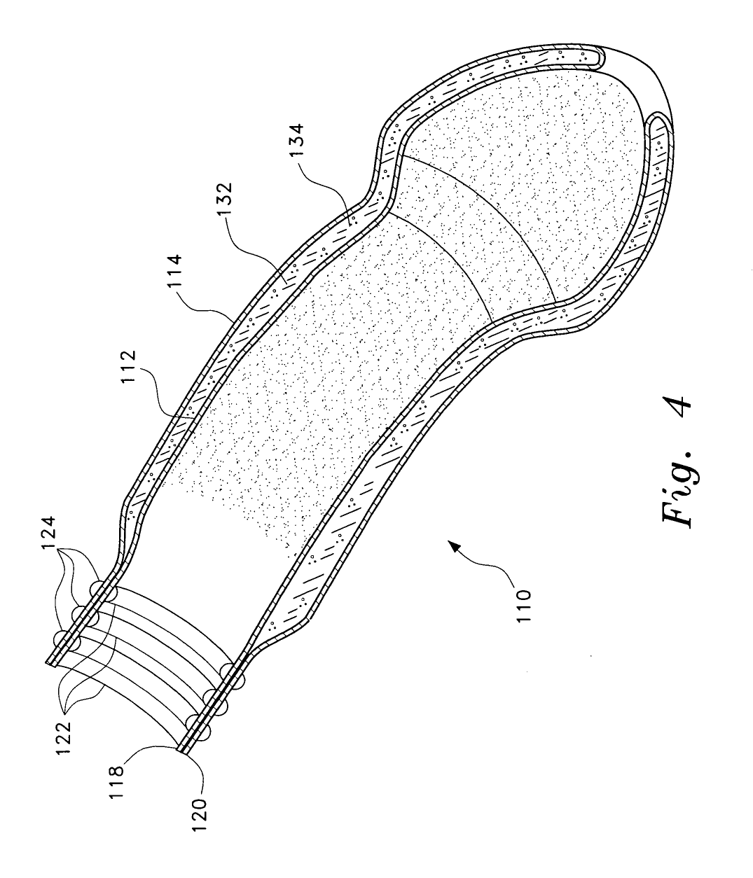

In the foreskin prosthesis embodiment 10 of FIGS. 1 through 3, the two sleeves 12 and 14 have different lengths and are separate from one another at their proximal open ends 18 and 20. FIG. 4 illustrates an alternative embodiment 110, in which the two sleeves 112 and 114 have the same lengths and their proximal ends 118 and 120 are imperviously sealed to one another. As the two sleeves 112 and 114 are formed as a single, unitary tube, the sealing of the two proximal ends 118 and 120 to one another defines an elongated annular volume 132 contained by the two sleeves 112 and 114. This volume 132 may be filled with a gel, viscous liquid, or other suitable substance 134 to provide further tactile insulation for the penis, and especially the glans installed therein. Such a foreskin prosthesis embodiment 110 may be manufactured of any suitable thin-walled, flexible, resilient, seamless material, so long as it is impervious to the passage of the liquid or gel substance 134 disposed therein. Other features provided in the embodiment of the prosthesis 10 of FIGS. 1 through 3, e.g., flocking, securing bands 122 and 124, etc., may be incorporated into the embodiment 110 of FIG. 4, as desired.

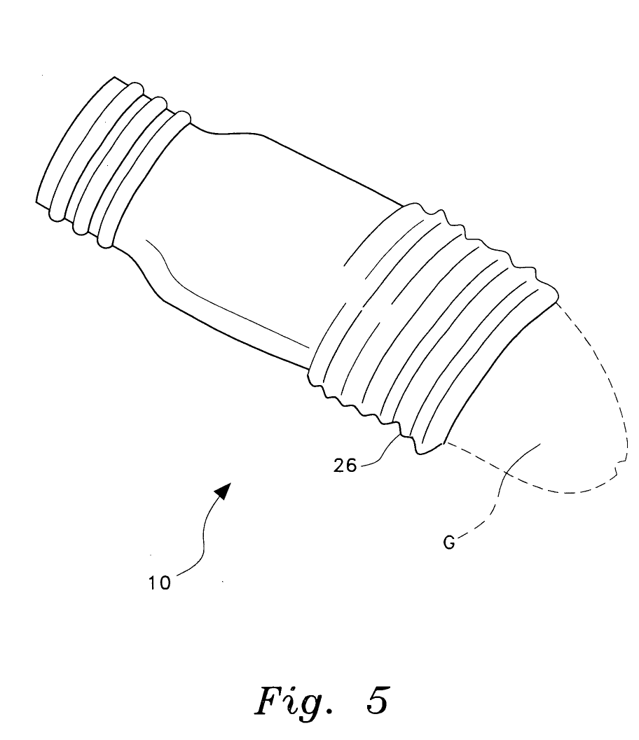

FIG. 5 of the drawings illustrates an advantageous feature of the present foreskin prosthesis in its various embodiments. In FIG. 5, the glans coverage portion 26 of the prosthesis 10 has been retracted or gathered generally about the corona of the glans G, exposing the glans. This function simulates the function of the natural foreskin, which is normally sufficiently pliable to allow it to be retracted or gathered behind the glans to expose the glans as desired. The selectively retractable and gatherable glans cover portion 26 is not limited to the prosthesis 10 embodiment, but is also a feature of the foreskin prosthesis embodiment 110 of FIG. 4 and other embodiments as well.

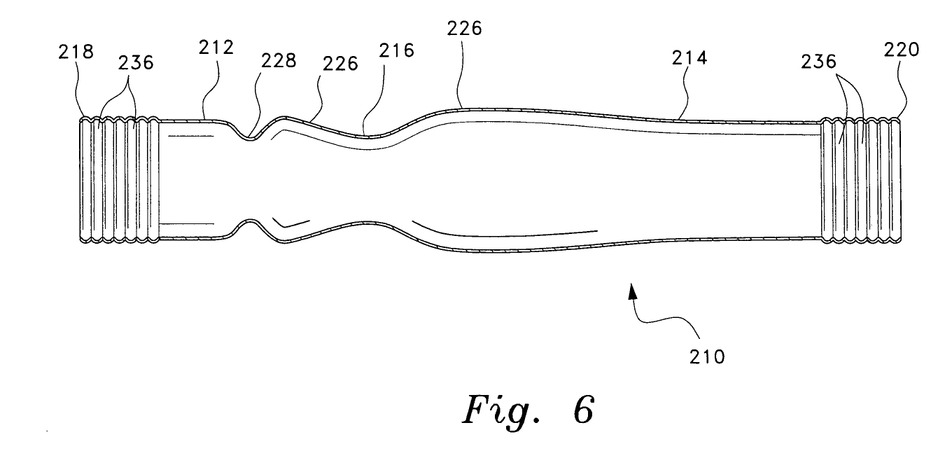

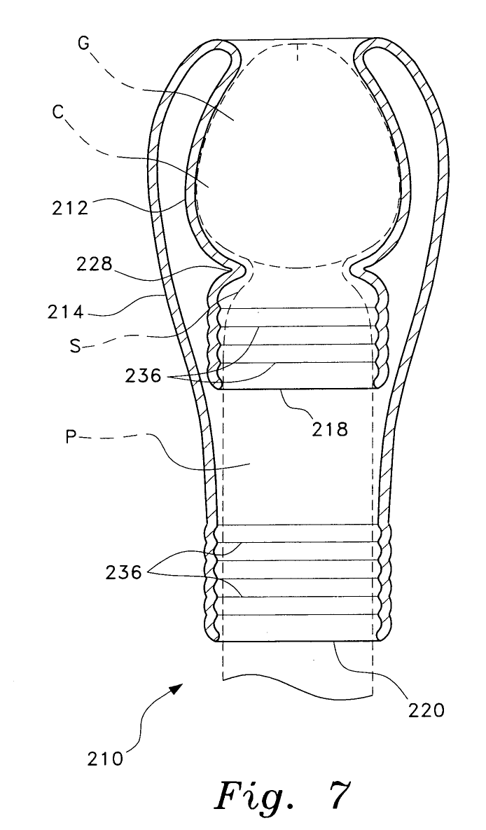

FIGS. 6 and 7 illustrate elevation views in section of still another variation on the embodiment 10 of FIGS. 1 through 3, with the prosthesis 210 of FIGS. 6 and 7 primarily differing in the specific configuration and local diameters of the inner sleeve portion 212. The foreskin prosthesis embodiment 210 of FIGS. 6 and 7 includes an inner sleeve portion 212 and an outer sleeve portion 214 joined at their common distal opening end 216. It will be noted that the diameter of the distal opening end 216 is relatively large in comparison to the open distal end 16 or 116 of the prostheses 10 and 110 of FIGS. 1 through 4. Such a larger distal open end 216 may facilitate the retraction or gathering of the glans coverage portion 226 about the corona C of the glans G when relatively inelastic materials are used for the prosthesis.

It will be further noted that the sulcus coverage area 228 of the prosthesis embodiment 210 has a relatively narrow diameter at the inner sleeve portion 212 in comparison to the generally uniformly expanding diameter of the outer sleeve portion 214 from the penile shaft P toward the glans G. This provides a close fit for the inner sleeve portion 212 of the device about the circumcised penile anatomy, while the gradually varying diameter of the outer sleeve portion more closely emulates the external shape of the natural foreskin. While the prosthesis 210 of FIGS. 6 and 7 is shown having two different lengths for the internal and external sleeve portions 212 and 214, with two separate proximal ends 218 and 220, it will be seen that the two portions may have equal lengths with their proximal ends sealed to one another to create an annular volume for gel or liquid, as in the embodiment 110 of FIG. 4, if so desired. Such a gel filled prosthesis with its relatively uniform outer diameter, closely emulates the natural foreskin in both physical configuration and function.

The foreskin prosthesis embodiment 210 of FIGS. 6 and 7 differs from the other embodiments in yet another respect as well, in that the securing means at the proximal ends 218 and 220 differ from the securing means used in other embodiments. In the embodiment 210 of FIGS. 6 and 7, the two proximal ends 218 and 220 are provided with a series of circumferential ribs 236, rather than the internal and external securing bands 22 and 24 of the embodiment 10 of FIGS. 1 through 3. Such retaining ribs 236 may be sufficient to provide more than adequate retention of the device, depending upon the nature of the material from which the device is formed and perhaps other factors. It will be seen that such ribs 236 may be provided on any of the other embodiments of the present foreskin prosthesis, and/or the retaining bands of other embodiments may be incorporated with the embodiment 210 of FIG. 6, if so desired.

The material from which the present foreskin prostheses are formed in their various embodiments can cause some difficulty in installation of the device unless the device is properly configured, particularly when a latex or plastic material is used. Moreover, where an internal retaining band(s) of high friction coefficient material is used, it can prove difficult to slide such material over the surface of the penis when applying the prosthesis.

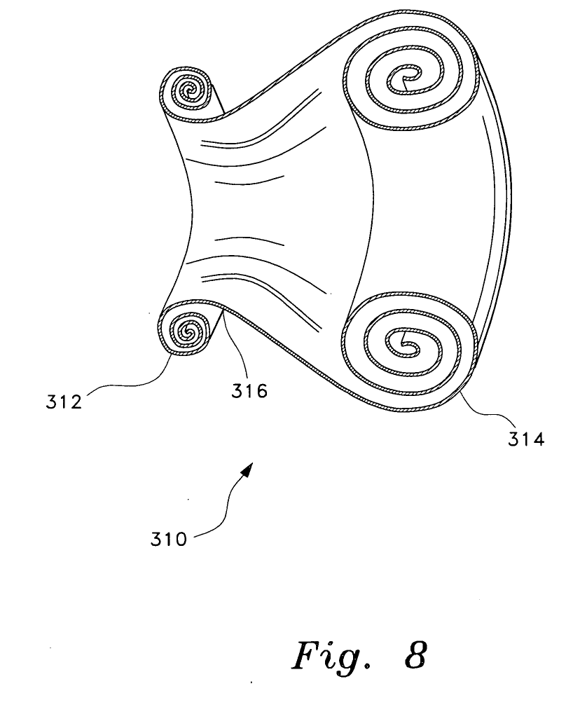

A solution to this problem may be provided by rolling the inner and outer sleeves, e.g., sleeves 312 and 314 of the embodiment 310 of FIG. 8, in opposite directions at the time of manufacture or packaging. The inner sleeve portion 312 is rolled circumferentially to the outside of the tube, allowing it to be applied over the penis by positioning the distal end of the penis within the externally rolled inner sleeve portion at the narrower diameter portion, which will form the distal end opening 316 when the device is installed. The externally rolled inner sleeve portion is then unrolled over the glans, sulcus, and penile shaft, as is conventionally known in applying a condom or many external male catheters.

The remaining outer sleeve portion 314 is then everted over the distal end of the penis, whereupon the rolled sleeve 314 is everted from an internally rolled configuration to an externally rolled configuration. The externally rolled configuration of the everted outer sleeve portion 314 allows it to be rolled over the glans, sulcus, and penile shaft in the same manner as used to don the internal sleeve portion 312. It will be seen that the above rolled configuration may be used with any of the various embodiments of the present foreskin prosthesis, as desired.

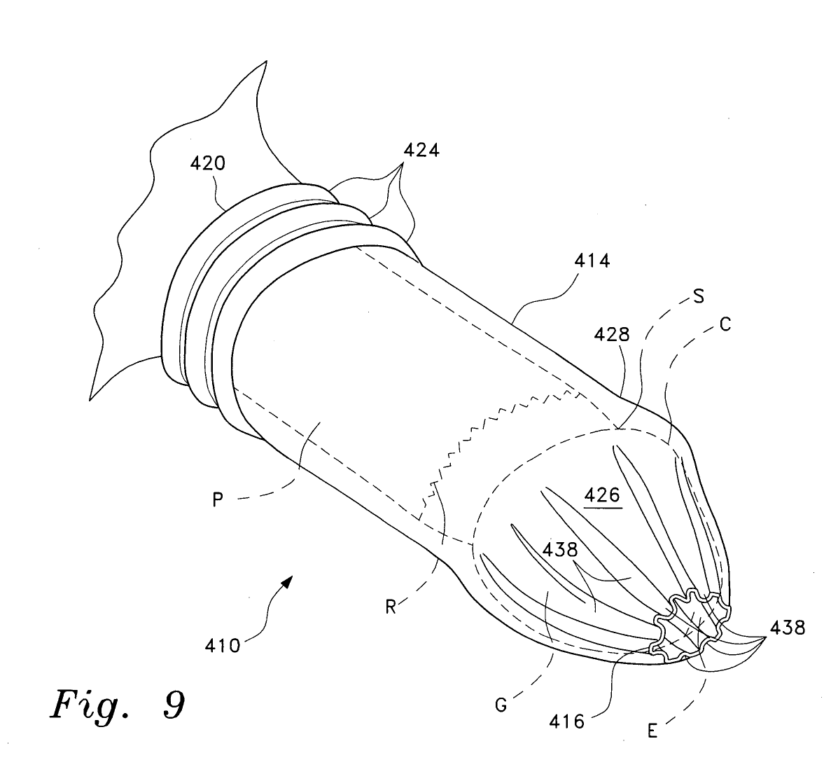

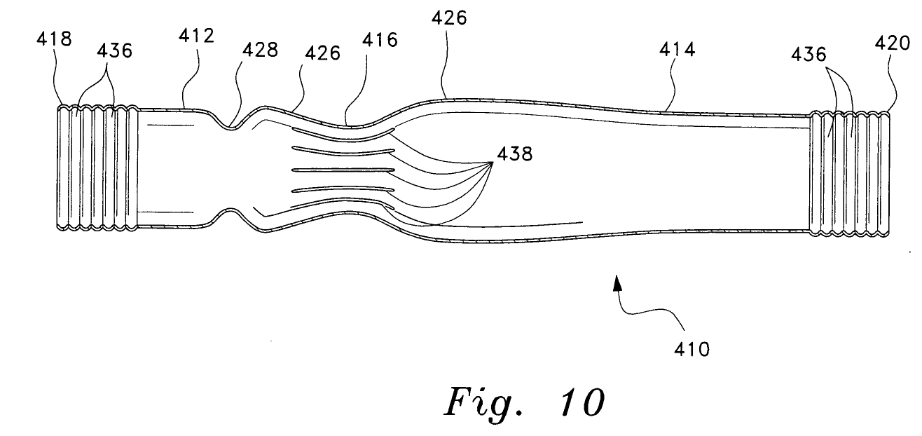

FIGS. 9 through 12 illustrate further embodiments of the present foreskin prosthesis, in which the portion of the device which covers the glans G is configured to facilitate its retraction and/or gathering back over the glans when so desired. The embodiment 410 of FIGS. 9 and 10 comprises an inner sleeve 412 and an outer sleeve 414 respectively having proximal ends 418 and 420, and a distal end opening 416 formed when the outer sleeve 414 is everted back over the inner sleeve 412. The opposite ends 418 and 420 may be provided with retaining ribs 436 in the manner of the embodiment 210 of FIG. 6, or other retaining means, if so desired. Preferably, the prosthesis 410 includes a relatively smaller diameter sulcus coverage area 428 and a glans coverage area 426 to either side of the portion 416 forming the end opening when the outer sleeve 414 is everted over the inner sleeve 412 during use.

The result is quite similar to the first embodiment 10 illustrated in FIGS. 1 through 3 of the drawings, and described in detail further above. However, the embodiment 410 of FIGS. 9 and 10 includes a modification to the glans coverage area and distal end opening portion 416. In the embodiment 410, a series of axially disposed folds or flutes 438 is formed across the area 416 which defines the end opening when the outer sleeve 412 is everted over the inner sleeve 414. As the material from which the present prosthesis is formed is quite thin and flexible, the axial folds or flutes 438 readily distend or spread circumferentially to fit over the relatively larger diameter glans G as the glans coverage portion 426 is withdrawn, gathered or retracted back over the glans G. The result is much like that shown in FIG. 5 of the drawings for the first embodiment 10, with any axial folds or flutes provided not being visible in this configuration due to their circumferential stretching and spreading around the larger diameter glans G. When the glans coverage portion or area 426 is again extended to cover the glans G, as shown in FIG. 9, the resilience of the material results in the folds or flutes 438 reforming to contract the end opening area 416 around the end E of the penis P, thereby providing complete coverage and protection for the otherwise exposed glans G.

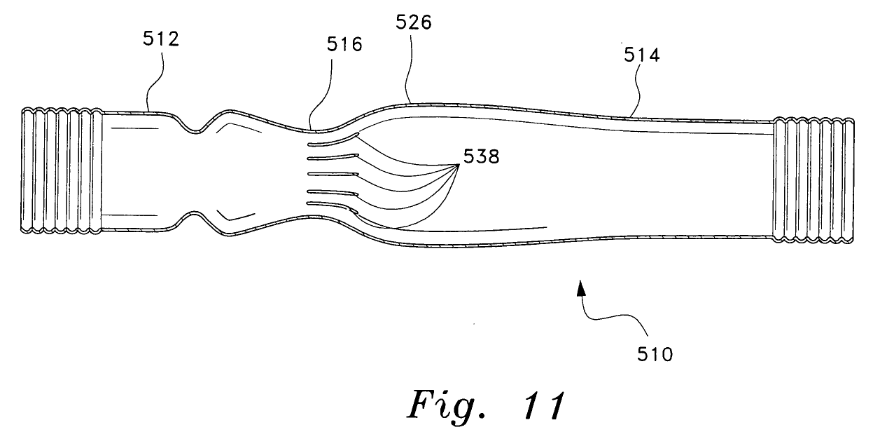

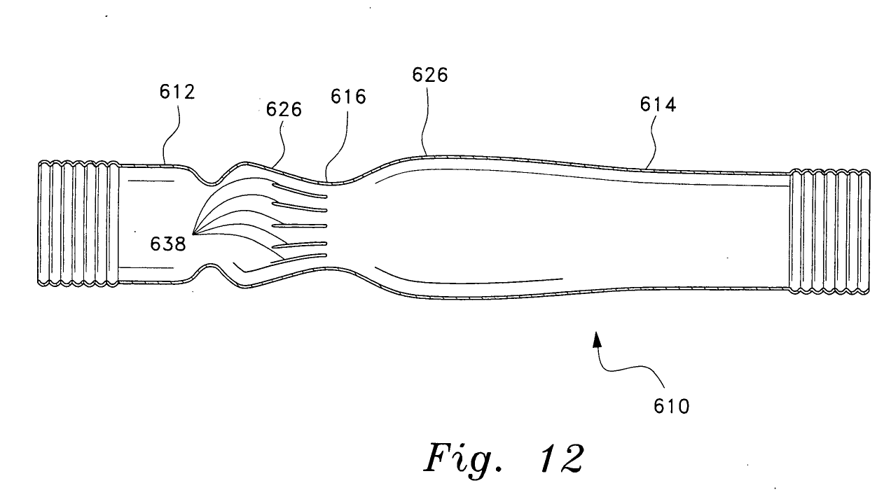

FIGS. 11 and 12 illustrate a pair of additional alternate embodiments 510 and 610 which differ slightly from the embodiment 410 of FIGS. 9 and 10. In FIG. 11, the folds or flutes 538 extend only from about the narrowest portion 516 of the device 510, i.e. that portion forming or defining the end opening when the outer sleeve 514 is everted back over the inner sleeve 512, along the glans coverage area or portion 526 of the outer sleeve 514. This configuration essentially reduces the restriction around the glans on the order of fifty percent, more or less, when the glans coverage portion 526 is retracted back from the end of the glans, with only the single layer of the glans coverage portion 526 of the inner sleeve 512 resulting in any noticeable restriction due to its lack of any axial folds therein.

In FIG. 12, the folds or flutes 638 extend only from about the narrowest portion 616 of the device 610, i.e. that portion forming or defining the end opening when the outer sleeve 614 is everted back over the inner sleeve 612, along the glans coverage area or portion 626 of the inner sleeve 612. This results in some circumferential restriction about the glans as a result of the outer sleeve portion of the glans coverage area 626 being devoid of the axial folds or flutes. While it is preferred that the axial folds or flutes be formed continuously along the glans coverage areas of both the inner and outer sleeve portions of the device, i.e. extending equally to both sides of the end opening area of the device as shown in the embodiment 410 of FIGS. 9 and 10, it will be appreciated that the more limited fold or flute configurations of the embodiments 510 and 610 of FIGS. 11 and 12 also serve to reduce the circumferential restriction when the prosthesis is retracted, although to a lesser degree than that provided by the more complete axial folds or flutes of the embodiment 410 of FIGS. 9 and 10.

In conclusion, the various embodiments of the foreskin prosthesis provide a workable solution to a problem experienced by vast numbers of men everywhere. The foreskin is a natural organ of the body, and its removal by circumcision can result in numerous problems, which were not considered in the past. In more recent times these problems have come to light in our more enlightened society, and men have realized that there can be numerous drawbacks to circumcision, both physical and psychological.

The various embodiments provide a reasonably realistic foreskin prosthesis for circumcised men who require such an article. The prosthesis is reasonably economical to purchase and may be worn for extended periods, with removal being at the option of the owner for bathing and sexual intercourse. The additional sensitivity of the penis, and particularly the glans, after the use of the prosthesis for some period of time can restore much sexual pleasure and enjoyment to men who previously felt that such activity and feelings were no longer parts of their lives. Accordingly, the present foreskin prosthesis will prove to be a most valuable addition for many men who have suffered circumcision in the past.

It is to be understood that the present invention is not limited to the embodiments described above, but encompasses any and all embodiments within the scope of the following claims.

I claim:

1. A foreskin prosthesis, comprising:
an inner sleeve; and
an outer sleeve, each of the sleeves being formed of a thin, flexible, resilient, seamless length of tubular material having a selectively retractable and gatherable glans coverage portion with an open distal end, and a penile shaft coverage portion with an open proximal end opposite the distal end, each of the sleeves being joined with one another at the distal end thereof to form a continuous, unbroken tube, at least one glans coverage portion further including a plurality of axially disposed folds therein, the outer sleeve being everted over the inner sleeve concentric with and surrounding the inner sleeve when the sleeves are applied over a human penis.

2. The foreskin prosthesis according to claim 1, wherein:
said inner sleeve is adapted for extending from the distal end thereof to the penile shaft adjacent the coronal sulcus; and
said outer sleeve is adapted for extending from the distal end thereof substantially to the base of the penis.

3. The foreskin prosthesis according to claim 1, wherein said inner sleeve and said outer sleeve are equal in length to one another and sealed to one another at their proximal ends, said inner sleeve and said outer sleeve defining an elongated annular volume therebetween, the foreskin prosthesis further comprising a fluid filling the annular volume.

4. The foreskin prosthesis according to claim 1, further including a softly flocked penis contact surface disposed upon at least said inner sleeve.

5. The foreskin prosthesis according to claim 1, further including at least one resilient securing band disposed circumferentially about the proximal end of at least said inner sleeve.

6. The foreskin prosthesis according to claim 1, wherein at least said inner sleeve has a configuration closely conforming to the glans, sulcus, and penile shaft of the penis.

7. The foreskin prosthesis according to claim 1, wherein said inner sleeve and said outer sleeve are formed of materials selected from the group consisting of latex, synthetic plastics, finely woven knitted fabrics, and finely woven braided fabrics.

8. A foreskin prosthesis for a human penis, comprising a single, continuous sleeve formed of a thin, flexible, resilient, seamless length of tubular material having a glans coverage portion with an open distal end and a penile shaft coverage portion with an open proximal end opposite the distal end, the sleeve having a narrow diameter center portion defining an inner portion adapted for covering the glans up to the penile shaft adjacent the coronal sulcus and an outer portion foldable back over the inner portion concentric therewith and closely fitting thereabout, at least a part of the glans coverage portion further including a plurality of axially disposed folds therein, the outer portion being adapted for covering the glans up to the base of the penis.

9. The foreskin prosthesis according to claim 8, wherein said glans coverage portion has a selectively retractable and gatherable configuration, said axially disposed folds distending circumferentially as said glans coverage portion is retracted and gathered over the glans.

10. The foreskin prosthesis according to claim 8, further including a softly flocked penis contact surface disposed upon at least said inner portion.

11. The foreskin prosthesis according to claim 8, further including at least one resilient securing band disposed circumferentially about the proximal end of at least said inner portion.

12. The foreskin prosthesis according to claim 8, wherein at least said inner portion has a configuration adapted for closely conforming to the glans, sulcus, and penile shaft of the penis.

13. The foreskin prosthesis according to claim 8, wherein said inner portion and said outer portion are formed of materials selected from the group consisting of latex, synthetic plastics, finely woven knitted fabrics, and finely woven braided fabrics.

14. A foreskin prosthesis, comprising:
a single, continuous sleeve formed of a thin, flexible, resilient, seamless length of tubular material having a glans coverage portion with an open distal end and a penile shaft coverage portion with an open proximal end opposite the distal end, at least a part of the glans coverage portion further including a plurality of axially disposed folds therein, the sleeve having an inner layer and an outer layer having proximal and distal ends joined to each other to define an elongated annular chamber between the layers, the sleeve being adapted for covering the glans at least up to the penile shaft adjacent the coronal sulcus; and
a fluid filling the annular chamber.

15. The foreskin prosthesis according to claim 14, wherein said glans coverage portion has a selectively retractable and gatherable configuration, said axially disposed folds distending circumferentially as said glans coverage portion is retracted and gathered over the glans.

16. The foreskin prosthesis according to claim 14, further including a softly flocked penis contact surface disposed upon at least said inner layer.

17. The foreskin prosthesis according to claim 14, further including at least one resilient securing band disposed circumferentially about the proximal end of at least said inner layer.

18. The foreskin prosthesis according to claim 14, wherein at least said inner layer has a configuration adapted for closely conforming to the glans, sulcus, and penile shaft of the penis.

19. The foreskin prosthesis according to claim 14, wherein said inner layer and said outer layer are formed of materials selected from the group consisting of latex and liquid impervious synthetic plastics.
